(19) United States
(12) Patent Application Publication
(10) Pub. No.: US 2004/0133187 A1
(43) Pub. Date: Jul. 8, 2004

(54) Methods and systems for providing orthogonally redundant monitoring in a sedation and analgesia system

(75) Inventor: Randall S. Hickle, Lubbock, TX (US)

Correspondence Address: HOGAN & HARTSON LLP, IP GROUP, COLUMBIA SQUARE, 555 THIRTEENTH STREET, N.W., WASHINGTON, DC, 20004, US

(73) Assignee: Scott Laboratories, Inc.

(21) Appl. No.: 10/677,483

(22) Filed: Oct. 3, 2003

Related U.S. Application Data
(60) Provisional application No. 60/415,522, filed on Oct. 3, 2002.

(51) Int. Cl. A61K 9/22

(52) U.S. Cl. 604/890.1

(57) ABSTRACT

The present invention includes a sedation and analgesia system having a high sensitivity and specificity for diagnostic and therapeutic algorithms, where the high sensitivity and specificity may be gained by providing multiple monitors for a single patient parameter. The invention also comprises multiple monitors for a single patient parameter, where the monitored data from each monitor is compared with that of the others by a controller in order to ascertain whether monitored data is reliable.

### CROSS REFERENCE TO RELATED APPLICATIONS

This application claims priority under 35 U.S.C. §119(e) to U.S. Provisional Patent Application No. 60/415,522, “Methods and Systems for Providing Orthogonally Redundant Monitoring in a Sedation and Analgesia System,” filed Oct. 3, 2002, which is hereby incorporated by reference.

### STATEMENT REGARDING FEDERALLY SPONSORED RESEARCH OR DEVELOPMENT

Not Applicable

### REFERENCE TO A “MICROFICHE APPENDIX”

Not Applicable

### BACKGROUND OF THE INVENTION

1. Field of the Invention

The present invention relates, in general, to orthogonal redundancy in clinical heuristics and, more particularly, to incorporating orthogonal redundancy into the monitoring features of a sedation and analgesia system.

2. Description of the Related Art

A sedation and analgesia system has been developed to provide patients undergoing painful, uncomfortable or otherwise frightening (anxiety inspiring) medical or surgical procedures with a means for receiving sedative, analgesic, and/or amnestic drugs safely in a way that reduces the risk of overmedication with or without the presence of a licensed anesthesia provider. Due to significant advances in technology, the sedation and analgesia system may be safer for use in hospital and ambulatory environments and may be operated by individuals other than trained anesthesiologists such as, for example, C.R.N.A.s, trained physicians, or other trained operators. The sedation and analgesia system has gone far to meet the needs of practitioners who are unable to schedule anesthesia providers for every procedure where safe and effective sedation and analgesia could substantially mitigate the effects of fear and pain. The advent of a sedation and analgesia system devoted to these purposes provides these individuals with a drug delivery system integrated into a patient monitoring system that decreases the cognitive and manual workload required with the operation of anesthesia machines, yet keeps the clinician in the loop of patient management. The clinician maintains ultimate decision making responsibility following a “clinician knows best” philosophy. This advanced technology allows for the sedation and analgesia system to be operated at drug level effects less than general anesthesia without an anesthesia provider, providing the patient with a cost-effective and readily available means of sedation, amnesia, and/or analgesia.

The sedation and analgesia system described in U.S. patent application Ser. No. 09/324,759 generally electronically integrates, for example, the delivery of one or more sedative, analgesic, and/or amnestic drugs, the delivery of positive airway pressure, decreases or increases in drug delivery, the delivery of oxygen, changes in drugs to, for example, an opioid antagonist, requests for additional information from patient monitors, and the triggering of alarms, with the electronic monitoring of one or more patient physiological conditions. application Ser. No. 09/324,759 was filed Jun. 3, 1999 and is incorporated herein by reference in its entirety. This system uses one or more sets of stored data-defining parameters reflecting patient and system states, the parameters being accessed through software to conservatively manage and correlate drug delivery to safe, cost effective, optimized values related to a conscious patient's vital signs and other physiological conditions.

The sedation and analgesia system has generally gone far to ensure patient safety by integrating patient monitoring with drug delivery, however, monitor failures, spurious monitored data, or other factors may cause the sedation and analgesia system to take potentially hazardous action, to fail to take action in critical situations, or to alarm unnecessarily. For example, a sedation and analgesia system may be monitoring a patient's heart rate with an electrocardiograph (ECG) when the ECG becomes erratic. Based on the single monitor, the sedation and analgesia system may signal an alarm indicating, for example, a dangerously low heart rate, when the erratic ECG data is actually spurious. A high frequency of false positive alarms may annoy clinicians and may result in less attention being given to truly life-threatening conditions.

### SUMMARY OF THE INVENTION

The present invention comprises a sedation and analgesia system with both high sensitivity and specificity for diagnostic and therapeutic algorithms. The highly sensitive system ensures that when a truly critical event occurs, the event is not missed. In a highly specific system, when an alarm signals an event, the alarm is representative of a truly critical situation, and not one that is based on spurious data. Providing a single monitor, such as an ECG, to monitor heart rate may result in the sedation and analgesia system having a low specificity, where if the single monitor provides spurious data, a false positive alarm may occur. In clinical settings with current physiology monitoring systems, false positive alarms occur commonly. The present invention provides a monitoring system that increases the specificity of the system while retaining a high degree of sensitivity.

The present invention includes a sedation and analgesia system having a high sensitivity and specificity, where the high sensitivity and specificity may be gained by providing multiple monitors for a single patient parameter, such as heart rate, for example. The invention also comprises multiple monitors for a single patient parameter, where the monitored data from each monitor is compared with that of the others by a controller in order to ascertain whether the monitored data is reliable. It is further advantageous to program the controller to take predetermined actions when monitors are in agreement as to patient condition and a different set of actions when the monitors are not. In conditions where monitors are not in agreement, the sedation and analgesia system of the present invention may immediately gather additional data, wait for a prescribed period of time to analyze additional data being gathered, provide “readily reversible” therapeutic interventions that are subsequently reversed in the event the triggered alarm state turns out to have been incorrect, provide an early intervention that is temporarily silent, or other algorithms to both diminish the presence of false negative (increased sensitivity) and false positive (increased specificity) alarms and to decrease the user annoyance and distraction imposed by false alarm conditions.

### BRIEF DESCRIPTION OF THE DRAWINGS

FIG. 1 illustrates a block diagram depicting one embodiment of a sedation and analgesia system in accordance with the present invention;

FIG. 2 illustrates one embodiment of an orthogonal redundancy system in accordance with the present invention;

FIG. 3 illustrates one embodiment of a method for providing orthogonal redundancy in sedation and analgesia system;

FIG. 4 illustrates a further embodiment of an orthogonal redundancy system in accordance with the present invention;

FIG. 5 illustrates a further embodiment of an orthogonal redundancy system in accordance with the present invention that comprises ascribing points to monitors integrated with sedation and analgesia system; and

FIG. 6 illustrates a further embodiment of a method for employing an orthogonally redundant system.

### DETAILED DESCRIPTION OF THE INVENTION

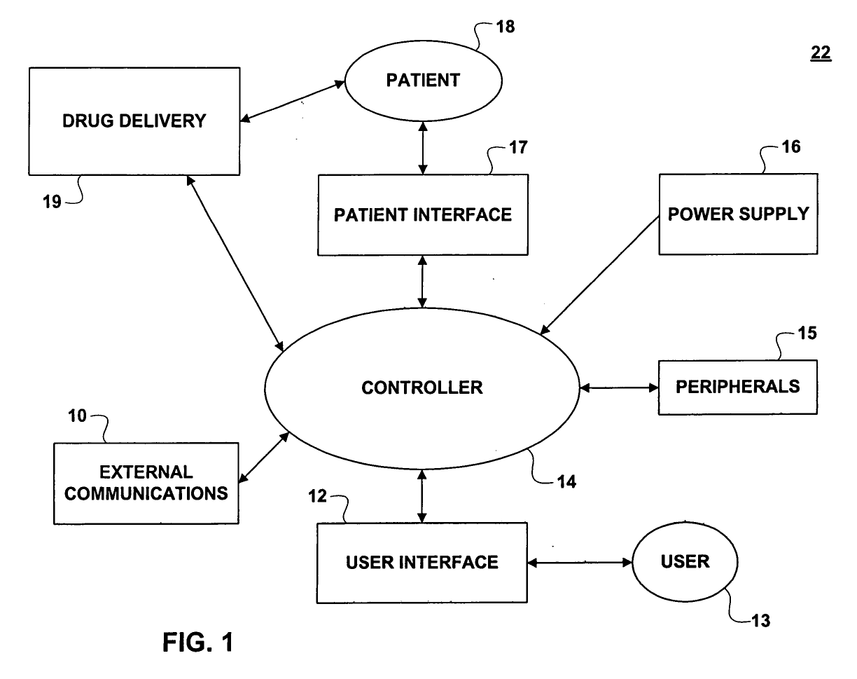

FIG. 1 illustrates a block diagram depicting one embodiment of a sedation and analgesia system 22 in accordance with the present invention having user interface 12, software controlled controller 14, peripherals 15, power supply 16, external communications 10, pressure delivery 11, patient interface 17, and drug delivery 19, where sedation and analgesia system 22 is operated by user 13 in order to provide sedation and/or analgesia to patient 18. An example of sedation and analgesia system 22 is disclosed and enabled by U.S. patent application Ser. No. 09/324,759, filed Jun. 3, 1999 and incorporated herein by reference in its entirety. Embodiments of user interface 12 are disclosed and enabled by U.S. patent application Ser. No. 10/285,689, filed Nov. 1, 2002, and incorporated herein by reference in its entirety.

Patient interface 17 includes two or more patient health monitors such as vital sign monitors and consciousness monitors including but not limited to non-invasive blood pressure monitors, pulse oximeters, capnometers, ECGs, patient consciousness assessment systems, ventilatory flow monitors, ventilatory pressure monitors, impedance plethysmogrophers (IPGs), gas analyzers, ventilatory temperature monitors, ventilatory humidity monitors, and acoustical monitors. The patient monitors of patient interface 17 may be electronically coupled to controller 14 and (through A-D converters, for example) provide feedback signals representing the patient's physiological condition. In one embodiment of the present invention, two or more patient monitors monitor a single patient parameter, such as heart rate, where the multiple monitoring of a single physiological parameter provides for orthogonal redundancy and a higher level of sensitivity and specificity. Controller 14 may compare the electronic feedback from patient interface 17 with data stored in a memory device, where such data may represent sets of one or more safe and undesirable patient physiological condition parameters such as, for example, safe and undesirable oxygen saturation conditions. These sets of data are collectively referred to as a safety data set, where data may include raw numbers (such as, for example, a measurement of electrical activity from an ECG) or information (such as, for example, a heart rate reading derived from the raw numbers). Based on the comparison, controller 14 may command a conservative application of drug delivery in accord with such parameters at safe, cost-effective optimized values.

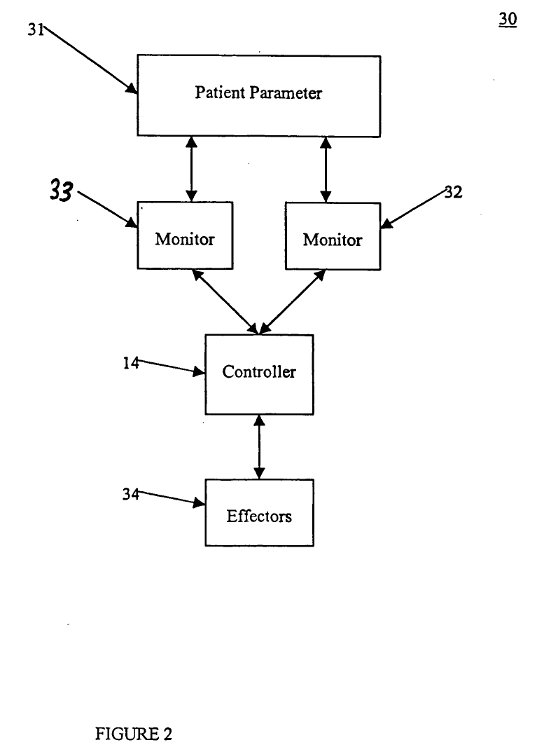

FIG. 2 illustrates one embodiment of an orthogonal redundancy system 30 in accordance with the present invention, where orthogonal redundancy system 30 comprises patient parameter 31, patient monitors 32 and 33, controller 14, and effectors 34. Patient parameter 31 may be any suitable patient parameter, such as heart rate or respiratory rate, where the parameter is a critical indicator of patient condition. Monitors 33 and 32 monitor patient parameter 31, where monitor 32 and monitor 33 gather data regarding patient parameter 31 independently of one another. Patient monitors 32 and 33 may be different types of monitors capable of monitoring patient parameter 31 in different way or they may be the same type of monitor but collect data independently of one another. For example, patient parameter 31 may be respiratory rate, where monitor 32 is a capnometer and monitor 33 is a pressure sensor. When patient parameter 31 is respiratory rate, monitors 32 and 33 may also be impedance plethysmographs (IPGs), ventilatory acoustical monitors, ventilatory humidity monitors, ventilatory temperature monitors, flow meters, gas analyzers, monitors that detect the changes of chest wall or abdominal diameter, pulse wave variation (PWV) monitors (where PWV monitors measure changes in cardiac output that correspond to respiration), or any other suitable respiratory monitor. Orthogonal redundancy system 30 further comprises any suitable number of monitors, where such monitors may be similar or dissimilar to one another. Monitored information, such as pressure and exhaled dioxide waveforms, may be transmitted to controller 14.

Controller 14 may be, for example, a microcontroller integrated with sedation and analgesia system 22 (FIG. 1), to which data from monitors 32 and 33 is transmitted. Controller 14 may be programmed to control effectors 34, where further embodiments of programmed heuristics will be further discussed herein. Controller 14 further comprises a safety data set as will be further discussed herein, where data from monitors 32 and 33 may be compared to data from the safety set in order to ascertain whether a patient is in a potentially critical situation. Effectors 34 may be any suitable control feature capable of ensuring patient safety and clinician awareness. Effectors 34 include, but are not limited to, drug decreases, drug increases, positive airway pressure changes, alarms, pre-alarms, oxygen delivery, triggers for additional data sampling from monitors 32 and 33, changes in drugs to, for example, carbon dioxide and opioid antagonists, and patient responsiveness queries. Effectors 34 may occur silently without alerting the attending clinician, they may be signaled by user interface 12, and/or they may require confirmation from the user before being initiated.

The multiple monitoring of a single patient parameter using separate monitoring techniques, herein known as orthogonal redundancy, allows for sedation and analgesia system 22 and the user to validate the data present on one monitor with that presented on another. For example, instead of alarming based on an erratic ECG reading, sedation and analgesia system 22 may look to pulse oximetry and non-invasive blood pressure (NIBP) to refute or affirm the data presented on the ECG. By using redundant monitoring systems concurrently, sedation and analgesia system 22 increases the specificity of the system by creating fewer false positive readings.

A sedation and analgesia system according to the present invention may make use of redundant capabilities of various monitors. For example, the fact that a pulse oximeter, whose main function typically is to provide blood saturation data and information, also provides heart rate data, which can then be compared to heart rate data and information from another monitor, such as an ECG monitor. Thus, the system can efficiently making use of existing data and information rather than increasing the cost of equipment by having redundant sub-systems for every monitored parameter.

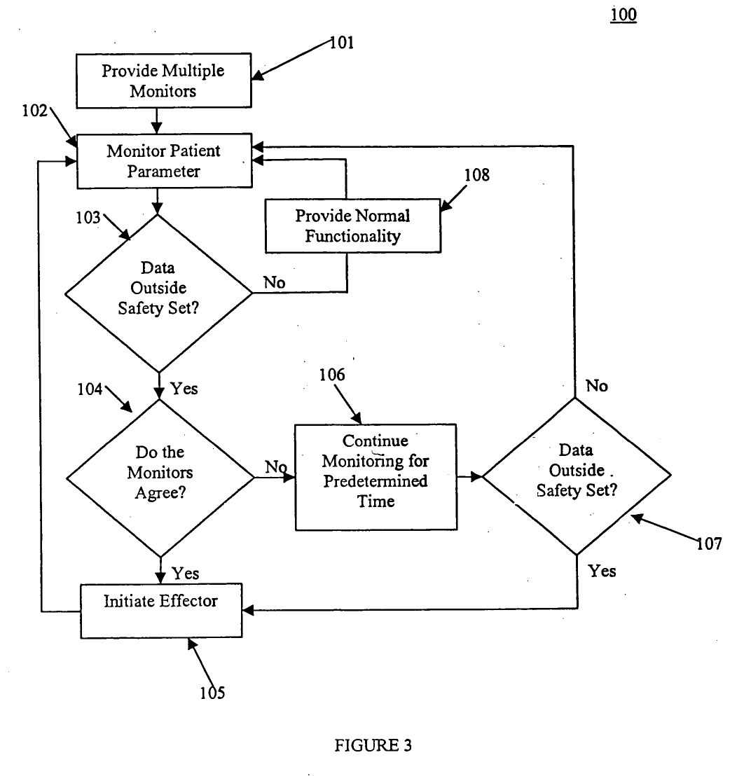

FIG. 3 illustrates one embodiment of a method 100 for providing orthogonal redundancy in sedation and analgesia system 22. Step 101 comprises providing multiple monitors of a single patient parameter 31 (FIG. 2), where the multiple monitors of step 101 may be monitors 32 and 33 (FIG. 2) or any other suitable number of patient monitors. Step 102 comprises monitoring the patient parameter 31 with the monitors, where the patient parameter may be, for example, heart rate, and where the monitors may be an ECG, a pulse oximeter, and a NIBP. Method 100 may continuously perform query 103 throughout the duration of a procedure, where query 103 comprises ascertaining whether any of the data transmitted from the patient monitors to controller 14 is outside the safety data set held in controller 14. If none of the monitors indicate that patient parameter 31 is outside of the data set, sedation and analgesia system 22 may proceed to step 108, where step 108 comprises providing normal sedation and analgesia system 22 functionality. Normal sedation and analgesia system 22 functionality may be pre-determined monitoring characteristics such as, for example, cycling the NIBP every three minutes and delivering a target concentration (for example, the target effect site concentration) of drug determined by the clinician. If a “yes” response has been given to query 103 at least one of the monitors indicates that the patient's monitored parameter is outside of the safety data set, and method 100 may proceed to query 104.

Query 104 comprises ascertaining whether the monitors associated with step 101 are in agreement as to whether patient parameter 31 is outside of the safety data set. If both monitors agree, where each monitor indicates that the patient is indeed outside of the safety data set, method 100 may proceed to step 105. Step 105 comprises initiating effectors associated with sedation and analgesia system 22 to attempt to alleviate the potentially dangerous status of patient parameter 31. The effectors associated with step 105 include, but are not limited to, decreasing the drug target concentration, increasing the drug target concentration, delivering positive airway pressure, triggering monitors associated with step 101 to cull more information, alarming, changing drugs from propofol to, for example, an opioid antagonist, delivering oxygen, and initiating pre-alarms based on trends that indicate a negative patient condition is imminent.

Controller 14 may be programmed to take any suitable action in accordance with step 105 to alleviate the cause for patient parameter 31 falling outside of the safety data set. In the case of heart rate and respiratory rate, this may be a result of overdose, where sedation and analgesia system 22 may, for example, decrease drug delivery, alert the attending clinician, and gather more data via stat monitoring systems, where examples of stat monitoring features incorporated into sedation and analgesia system heuristics are disclosed in application Ser. No. 09/324,759. While step 105 is active, method 100 may loop back to step 102, where if method 100 proceeds to step 108, the activated effector of step 105 may be discontinued. Step 108 may further comprise requiring a clinician to confirm a return to normal functionality following the initiation of an effector associated with step 105.

Returning to query 104, if the monitors associated with step 101 are not in agreement, where at least one shows that patient parameter 31 is outside of the safety data set, method 100 may proceed to step 106. Step 106 comprises gathering additional information from patient monitors, where instead of alarming at the first sign of erratic data, sedation and analgesia system 22 may wait for a predetermined period of time to analyze additional data before alerting a clinician. After, for example, 15 seconds of additional monitoring, method 100 may proceed to query 107.

Query 107 comprises ascertaining whether the monitors associated with step 101 indicate that patient parameter 31 is still outside of the safety data set following step 106. If patient parameter 31 is still outside of the safety data set on at least one of the patient monitors or a majority of the monitors, method 100 may proceed to step 105; if all monitors are now in agreement that patient parameter 31 is outside the safety data set, the effectors of step 105 may proceed as illustrated earlier. However, step 105 further comprises initiating a separate protocol if the patient monitors are not in agreement, yet at least one monitor indicates that patient parameter 31 is outside of the safety data set. For example, if patient parameter 31 is respiratory rate and is being monitored by capnometry and a pressure monitor, where the pressure monitor indicates data outside of the safety data set and the capnometer does not, then sedation and analgesia system 22 may alert the clinician but initiate no other effector until confirmation of such is received. Keeping the clinician in the loop may avoid unnecessary effectors that might, for example, bring the patient out of a state of sedation, where monitoring problems may still be effectively evaluated and/or corrected. Returning to query 107, if data from all monitors is no longer outside of the safety data set, method 100 may proceed to step 102. Method 100 may be terminated at any suitable time during a medical procedure, where any action may be immediately truncated by a clinician's command.

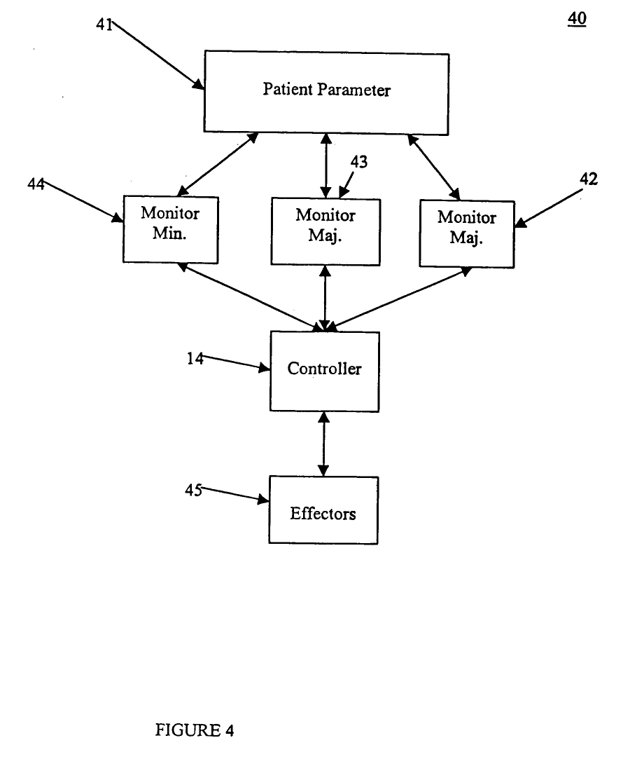

FIG. 4 illustrates a further embodiment of an orthogonal redundancy system 40 in accordance with the present invention. Orthogonal redundancy system 40 comprises a patient parameter 31, a minor monitor 44, major monitors 42 and 43, a controller 14, and effectors 45. Patient parameter 31 may be a patient's heart rate, respiratory rate, or another critical physiological parameter. Minor monitor 44 may be any suitable monitor that, for example, provides data regarding patient parameter 31, but may be prone to artifact, disturbance, and/or otherwise is not always a reliable indicator of patient condition. If patient parameter 31 is respiratory rate, then minor monitor 44 may be an acoustical ventilatory monitor, where such monitors commonly provide spurious data. Major monitors 42 and 43 may be more reliable monitoring devices such as, but not limited to, capnometers, pressure monitors, flow meters, and gas analyzers. It is contemplated that monitors considered to be the most accurate in a procedure may vary from procedure to procedure, however, it may be beneficial to provide less accurate monitors, such as acoustical monitors, in cooperation with more accurate monitors, that still add relevant information to a case.

Orthogonal redundancy system 40 may be operated in the fashion illustrated in method 100, however only major monitors 43, 42 may be considered by sedation and analgesia system in its conservative decision making. Orthogonal redundancy ensures that multiple monitors monitor a single physiological characteristic in order to ensure that data processed by controller 14 and presented to the clinician is representative of true patient condition. Though greater numbers of monitors of a single patient parameter may provide added information to a clinician if all monitors are providing accurate data or information, certain monitors may be too prone to artifact to integrate directly in routine operation into the conservative decision making processes of sedation and analgesia system 22. With this in mind, orthogonal redundancy system 40 comprises the addition of minor monitors 44 that may be, for example displayed to a clinician via user interface 12, however, they are not integrated with major monitors 42 and 43 in the conservative decision making processes of the system. The present invention comprises the addition of any suitable major monitors and minor monitors, where some of such monitors are integrated into the decision making processes of sedation and analgesia system 22 while others may simply just present data to the clinician.

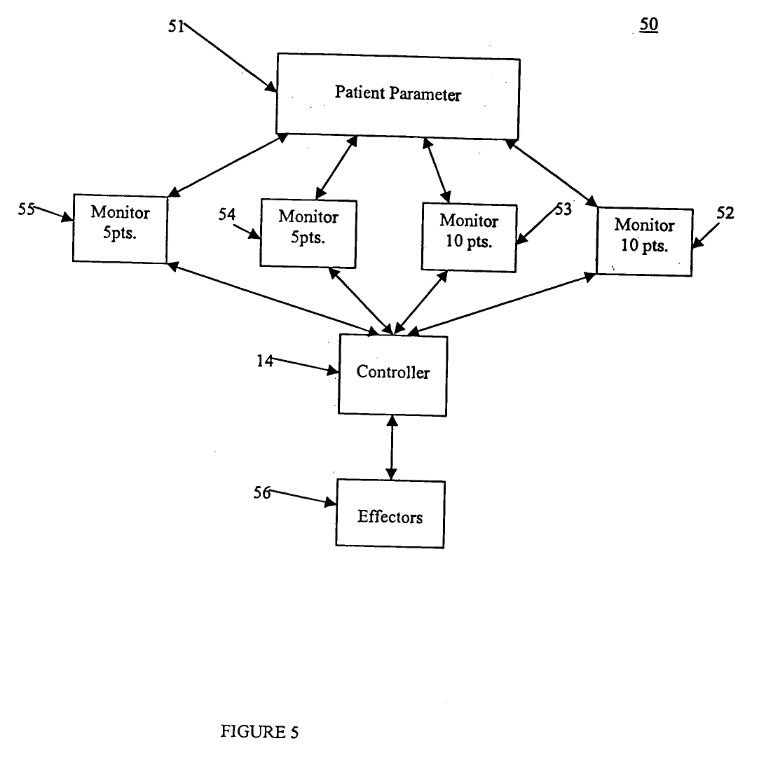

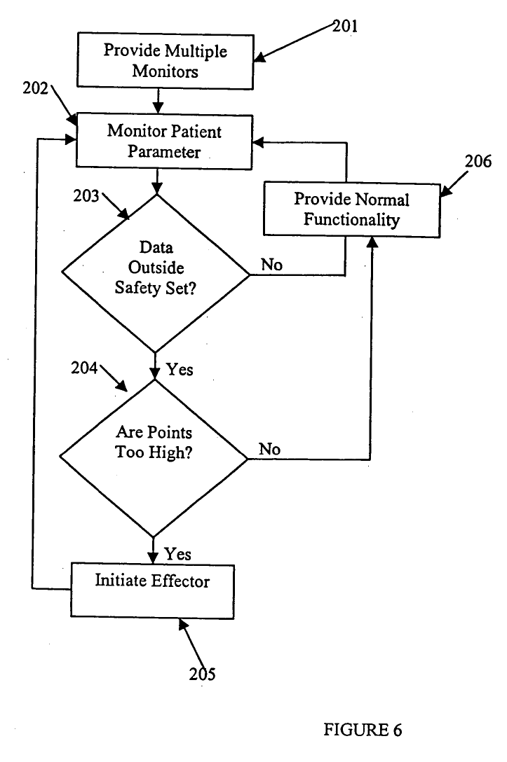

FIG. 5 illustrates a further embodiment of an orthogonal redundancy system 50 in accordance with the present invention that comprises ascribing points or weights to monitors integrated with sedation and analgesia system 22. Such points indicate to sedation and analgesia system 22 the criticality of the data received from each monitor. For example monitors 54 and 55 may be considered less critical and/or accurate than monitors 52 and 53, and accordingly, monitors 52 and 53 may be designated 5 point monitors while monitors 54 and 55 may be designated 10 point monitors. All four monitors (any suitable number of monitors may be used) may monitor the same patient parameter 51, where patient parameter 51 may be, for example, heart rate or respiratory rate. Monitors 52, 53, 54 and 55 communicate with controller 14 of sedation and analgesia system, where controller 14 initiates effectors 56 based on its incorporated programming. The heuristic method that makes use of the point system of orthogonal redundancy system 50 will be discussed further with respect to FIG. 6. It is further contemplated that any suitable point system or means of classifying the importance of orthogonally redundant monitors is in accordance with the present invention.

FIG. 6 illustrates one embodiment of a method 200 for employing orthogonally redundant system 50 (FIG. 5), where step 201 comprises providing multiple monitors 52, 53, 54 and 55 where such monitors are ascribed point values as to their importance and/or accuracy in monitoring patient parameter 51. If, for example, patient parameter 51 is respiratory rate, monitor 55 may be an acoustical monitor and monitor 54 may be a ventilatory humidity monitor, where such monitors are ascribed 5 points, lower than that of monitors 52 and 53, because of their tendency to provide spurious data. Monitor 53 may be a capnometer and monitor 52 may be a ventilatory pressure monitor, where such monitors are ascribed 10 points, higher than that of monitors 54 and 55, because of their greater significance and/or accuracy in monitoring patient parameter 51.

Step 202 comprises using multiple monitors 52, 53, 54 and 55 to monitor a selected patient parameter. Query 203 comprises ascertaining whether any of monitors 52, 53, 54 and 55 indicate data outside of a safety data set. If none of the monitors indicate data representative of a potentially dangerous patient situation, method 200 may proceed to step 206. Step 206 comprises maintaining normal functionality such as, for example, a target concentration in the absence of alarms, oxygen delivery, and positive airway pressure administration. Step 206 may continually loop back to step 202 to ensure that patient parameter 51 remains within acceptable bounds throughout the duration of the procedure. If at least one of monitors 52, 53, 54 and 55 indicates data outside of the safety data set, method 200 may proceed to query 204.

Query 204 comprises ascertaining whether the ascribed point values of monitors indicating a potentially dangerous patient condition add up to a number greater than a pre-determined threshold. For example, controller 14 may be programmed to, for example, alarm and discontinue drug delivery, in the event that the point values of the monitors displaying potentially critical data add up to a number of 15 or greater. Where, for example, if monitor 55 (a 5 point monitor) and monitor 53 (a ten point monitor) both indicate data outside the safety data set, then the pre-determined point threshold will have been met and sedation and analgesia system 22 will alarm and discontinue drug delivery. If however, monitor 55 and monitor 54 (both 5 point monitors) indicate data outside the safe data set, then the pre-determined point threshold will not have been met and sedation and analgesia system 22 may continue normal functionality in accordance with step 206.

Giving weight to the information received from more reliable and/or more significant monitors may allow for sedation and analgesia system 22 to more correctly ascertain the condition of patient parameter 51. Since monitors 52, 53, 54 and 55 will be monitoring the same patient parameter, an actual change in patient parameter 51 should be exhibited in all four monitors. If such a change takes place in only one of the illustrated monitors, where the other monitors monitoring the same patient parameter do not detect the same change, it is likely that the monitor transmitting data different from that of the others is inaccurate. In order to provide sedation and analgesia system 22 and the clinician with the most accurate data possible the more significant and accurate monitors may be given more points or weight in the conservative decision making process.

Step 205 comprises initiating effectors associated with sedation and analgesia system 22 to attempt to alleviate the potentially dangerous status of patient parameter 31. The effectors associated with step 205 include, but are not limited to, decreasing the drug target concentration, increasing the drug target concentration, delivering positive airway pressure, triggering monitors associated with step 201 to cull more information, alarming, changing drugs from propofol to, for example, an opioid antagonist, delivering oxygen, and initiating pre-alarms based on trends that indicate a negative patient condition is imminent.

Controller 14 may be programmed to take any suitable action in accordance with step 205 to alleviate the cause for patient parameter 31 falling outside of the safety data set on enough monitors to exceed the pre-determined point threshold. In the case of heart rate and respiratory rate, a negative patient condition may be a result of overdose, where sedation and analgesia system 22 may, for example, decrease drug delivery, alarm the attending clinician, and gather more data via stat monitoring systems. While step 205 is active, method 200 may loop back to step 202, where if method 200 proceeds to step 206, the activated effector of step 205 may be discontinued. Step 206 may further comprise requiring a clinician to confirm a return to normal functionality following the initiation of an effector associated with step 205.

The present invention comprises employing orthogonal redundancy to monitor any suitable patient or sedation and analgesia system parameter. It is further contemplated that technical elements of sedation and analgesia system 22 may employ orthogonal redundancy, where various system features such as, for example, software functionality, may be monitored by various redundant independent monitoring systems that function in accordance with the methods of the present invention. The present invention comprises any suitable combination of effectors, monitors, and monitored patient parameters necessary to ensure patient safety. The present invention further comprises initiating different effectors or different levels of effector initiation to overcome negative patient situations based on how severe the patient's condition is determined to be. Examples of such various thresholds are disclosed in application Ser. No. 09/324,759, where any suitable thresholds for any suitable effectors, monitors, and patient parameters are in accordance with the present invention.

While exemplary embodiments of the invention have been shown and described herein, it will be obvious to those skilled in the art that such embodiments are provided by way of example only. Numerous insubstantial variations, changes, and substitutions will now be apparent to those skilled in the art without departing from the scope of the invention disclosed herein by the Applicants. Accordingly, it is intended that the invention be limited only by the spirit and scope by the claims as they will be allowed.

1. A sedation and analgesia system, comprising:
two or more patient health monitor devices adapted so as to be coupled to a patient and so as to each generate a signal reflecting one or more physiological conditions of the patient wherein the operating principle of each of said monitors is different;
a user interface;
a drug delivery controller supplying one or more drugs to the patient;
a memory device storing a safety data set reflecting safe and undesirable parameters of at least one of said monitored patient physiological conditions;
one or more effector for ensuring patient safety and clinician awareness; and
an electronic controller interconnected with the patient health monitors, the user interface, the drug delivery controller, the memory device storing the safety data set, and the effector, wherein said electronic controller receives said signals and in response to said signals controls the effector in accordance with the safety data set.

2. The sedation and analgesia system of claim 1, wherein the patient health monitor devices are of different types.

3. The sedation and analgesia system of claim 2, wherein the patient health monitor devices each generate a signal reflecting a similar physiological condition of the patient.

4. The sedation and analgesia system of claim 3, wherein at least one of said patent health monitor devices provides high sensitivity and at least one other patient health monitor device provides high specificity.

5. The sedation and analgesia system of claim 1, wherein the monitors gather data regarding a physiological condition of the patient independently of one another.

6. The sedation and analgesia system of claim 1, wherein the patient health monitoring devices comprise two or more major monitors and at least one minor monitor, said major monitors being integrated into the decision making processes of the sedation and analgesia system and said minor monitors presenting data to the clinician.

7. The sedation and analgesia system of claim 1, wherein at least some of said patient health monitor devices are ascribed point values as to at least one of their importance and accuracy in monitoring a patient parameter.

8. The sedation and analgesia system of claim 1, wherein said effector includes at least one of decreasing a drug target concentration, increasing a drug target concentration, delivering positive airway pressure, triggering said monitors to cull more information, alarming, changing drugs, delivering oxygen, and initiating pre-alarms based on trends that indicate an imminent negative patient condition.

9. A sedation and analgesia system, comprising:
first means for monitoring health of a patient,
second means for monitoring health of said patient, wherein said second means is different from said first means and wherein each of said first and second monitoring means generate a signal reflecting one or more physiological conditions of the patient;
a user interface;
a drug delivery controller supplying one or more drugs to the patient;
a memory means for storing a safety data set reflecting safe and undesirable parameters of at least one of said monitored patient physiological conditions;
one or more effector for ensuring patient safety and clinician awareness; and
an electronic controller interconnected with the monitoring means, the user interface, the drug delivery controller, the memory means, and the effector, wherein said electronic controller receives said signals and in response to said signals controls the effector in accordance with the safety data set.

10. A method for providing orthogonal redundancy in sedation and analgesia system, comprising:
providing multiple monitors of a single patient parameter, wherein said monitors transmit patient data regarding said parameter;
monitoring the patient parameter with the monitors;
ascertaining whether any of the data transmitted from the patient monitors is outside a predetermined safety data set;
if none of the data is outside of the safety data set, providing normal sedation and analgesia system functionality;
if at least some of the data is outside of the safety data set, ascertaining whether the monitors are in agreement as to whether the data is outside of the safety data set; and
if the monitors are in agreement that data is outside the safety data set, initiating effectors associated with sedation and analgesia system.

11. The method of claim 10, further comprising the steps of:
if the monitors are not in agreement that data is outside of the safety data set, gathering additional information from patient monitors, and ascertaining whether the data from at least one monitor remains outside of the safety data set; and
if the monitors remain not in agreement that data is outside of the safety data set, initiating a separate predetermined protocol.

12. The method of claim 11, wherein said separate predetermined protocol comprises alerting a clinician.

13. The method of claim 12, wherein confirmation of said clinician is required to initiate an effector.

14. The method of claim 10, wherein said effectors include at least one of decreasing a drug target concentration, increasing a drug target concentration, delivering positive airway pressure, triggering said monitors to cull more information, alarming, changing drugs, delivering oxygen, and initiating pre-alarms based on trends that indicate an imminent negative patient condition.

15. A method for employing an orthogonally redundant system for use with a sedation and analgesia system, comprising:
providing multiple monitors, wherein such monitors are ascribed point values as to at least one of their importance and accuracy in monitoring a patient parameter;
monitoring the patient parameter with the monitors;
ascertaining whether any of the data transmitted from the patient monitors is outside a predetermined safety data set; and
if none of the data is outside of the safety data set, providing normal sedation and analgesia system functionality.

16. The method of claim 15, further comprising the steps of:
if at least some of the data is outside the safety data set, ascertaining whether the ascribed point values of monitors indicating a potentially dangerous patient condition add up to a number greater than a pre-determined threshold; and
if the sum of the ascribed point values exceed a predetermined value, initiating effectors associated with the sedation and analgesia system.

17. The method of claim 16, wherein said effectors include at least one of decreasing a drug target concentration, increasing a drug target concentration, delivering positive airway pressure, triggering said monitors to cull more information, alarming, changing drugs, delivering oxygen, and initiating pre-alarms based on trends that indicate an imminent negative patient condition.
